(19) United States
(12) Patent Application Publication
(10) Pub. No.: US 2024/0285923 A1
(43) Pub. Date: Aug. 29, 2024

(54) SHEATH HUB WITH INBUILT TIGHTENING PORT FOR CATHETER POSITION LOCKING

(71) Applicants: Boston Scientific Medical Device Limited, Galway (IE); Boston Scientific Scimed, Inc., Maple Grove, MN (US)

(72) Inventors: Neeraj Kumar Sharma, Delhi (IN); Sumit Agrawal, Gurgaon (IN); Qian Liu, Plymouth, MN (US); Rupesh Kumar Pahwa, Gurugram (IN)

(73) Assignees: Boston Scientific Medical Device Limited, Galway (IE); Boston Scientific Scimed, Inc., Maple Grove, MN (US)

(21) Appl. No.: 18/581,892

(22) Filed: Feb. 20, 2024

Related U.S. Application Data
(60) Provisional application No. 63/447,707, filed on Feb. 23, 2023.

(51) Int. Cl. A61M 39/06; A61M 25/00; A61M 25/01; A61M 25/02; A61M 39/02

(52) U.S. Cl. CPC A61M 39/0613; A61M 25/0097; A61M 2025/0166; A61M 2025/0286; A61M 2039/0258; A61M 2039/0288

(57) ABSTRACT

A valve hub assembly for use with a sheath includes a valve hub with a proximal end opposite a distal end, a first arm defining a lumen, and a second arm defining a lumen. A tightening port is arranged at the proximal end of the valve hub. The tightening port includes a hub cap engaged with the tightening port, a radially expandable seal positioned in a lumen of the hub cap, a pusher engaged with the radially expandable seal and at least partially disposed within the hub cap, and a lock nut arranged around the pusher and configured for pressing of the pusher against the radially expandable seal.

### CROSS-REFERENCE TO RELATED APPLICATIONS

This application claims the benefit of U.S. Provisional Patent Application Ser. No. 63/447,707, filed Feb. 23, 2023, which is incorporated herein by reference.

### TECHNICAL FIELD

The present disclosure relates to a hub of a sheath. More specifically, the present disclosure relates to a hub having an inbuilt tightening port that may be used to secure a medical device, such as a catheter, within the hub and thus fix the position of the catheter with respect to the hub and sheath.

### BACKGROUND

In various procedures for delivering intravascular medical devices, a sheath is inserted into a blood vessel of a patient, for example a femoral artery, and medical devices extend through the sheath and into the patient's vasculature. In various instances, the medical devices include catheters or other devices such as a blood pump. A hub may be incorporated at a proximal end of the sheath to reduce blood leakage as devices are being inserted, positioned, and removed. In various instances, there may be a desire to fix the positioning of the medical device within the sheath and vasculature. Additionally, there may be a desire to reposition the medical device within the vasculature. Thus, there is a need for improved tightening mechanisms for securing the medical device, such as a catheter, within the sheath and vasculature that also allow repositioning of the medical device in the vasculature.

### SUMMARY

In Example 1, a valve hub assembly includes a valve hub and a tightening port. The valve hub includes a proximal end opposite a distal end, a first arm defining a first lumen, and a second arm defining a second lumen. The tightening port is arranged at the proximal end of the valve hub and includes: a hub cap engaged with the tightening port, the hub cap defining a third lumen for receiving a radially expandable seal; a pusher engaged with the radially expandable seal and at least partially disposed within the hub cap; and a lock nut arranged around the pusher and configured for tightening of the pusher against the radially expandable seal.

In Example 2, the valve hub assembly of Example 1, wherein the tightening port further includes a sleeve holder engaged with the pusher.

In Example 3, the valve hub assembly of Example 2, wherein the tightening port further includes a sleeve gripper capable of engagement with the sleeve holder.

In Example 4, the valve hub assembly of any of the preceding Examples, further including a suture pad engaged with the distal end of the valve hub.

In Example 5, the valve hub assembly of any of the preceding Examples, wherein the tightening port further includes a primary seal arranged within the hub cap and positioned adjacent the first lumen.

In Example 6, the valve hub assembly of any of the preceding Examples, wherein the hub cap comprises a first portion, a second portion, and a transition wall defining a transition between the first portion and the second portion, and wherein the radially expandable seal is arranged within the first portion and adjacent the transition wall.

In Example 7, the valve hub assembly of Example 6, wherein the lock nut is configured to axially compress the pusher against the radially expandable seal such that the radially expandable seal expands radially to seal against the inner surface of the hub cap.

In Example 8, the valve hub assembly of any of the preceding Examples, wherein the radially expandable seal is a Tuohy seal.

In Example 9, the valve hub assembly of any of the preceding Examples, wherein the hub cap has a plurality of protrusions extending from a proximal end of the hub cap, the valve hub has a collar with a plurality of openings, and the plurality of protrusions of the hub cap are engaged with the plurality of openings such that rotation of the hub cap relative to the valve hub is inhibited.

In Example 10, a method for positioning and securing the positioning of a medical device includes: assembling a valve hub onto a sheath, the valve hub having a proximal end and a distal end; arranging a tightening port at the proximal end of the valve hub, the tightening port including a hub cap defining a first lumen for receiving a radially expandable seal, a pusher engaged with the radially expandable seal and at least partially disposed within the hub cap, and a lock nut arranged around the pusher and the hub cap for compressing the pusher against the radially expandable seal; delivering the medical device through the valve hub and the sheath; verifying the position of the medical device; tightening the tightening port; and securing a sterile sleeve using the tightening port.

In Example 11, the method of Example 10, wherein the tightening the tightening port includes actuating the lock nut to move the pusher against the radially expandable seal.

In Example 12, the method of Example 11, wherein the pusher causes radial expansion of the radially expandable seal to cause sealed engagement between the radially expandable seal and the hub cap.

In Example 13, the method of any one of Examples 10-12, wherein the hub cap has a plurality of protrusions extending from a proximal end of the hub cap, the valve hub has a collar with a plurality of openings, and the plurality of protrusions of the hub cap are engaged with the plurality of openings such that rotation of the hub cap relative to the valve hub is inhibited.

In Example 14, the method of any one of Examples 10-13, wherein the verification of the position of the medical device is conducted via fluoroscopy.

In Example 15, the method of any one of Examples 10-14, wherein the securing of the sterile sleeve with the tightening port further includes placing the sterile sleeve over a sleeve holder of the tightening port and arranging a sleeve gripper over the sterile sleeve and the sleeve holder.

In Example 16, a valve hub assembly for use with a sheath includes a valve hub and a tightening port. The valve hub includes a proximal end opposite a distal end, a first arm defining a first lumen, and a second arm defining a second lumen. The tightening port is arranged at the proximal end of the valve hub and includes: a hub cap engaged with the proximal end of the valve hub, the hub cap defining a third lumen for receiving a radially expandable seal; a pusher engaged with the radially expandable seal and at least partially disposed within the hub cap; and a lock nut arranged around the pusher and configured for tightening of the pusher against the radially expandable seal.

In Example 17, the valve hub assembly of Example 16, further including a sleeve holder for engaging the pusher without engaging the lock nut, wherein engagement of the sleeve holder and the pusher is configured such that rotation of the lock nut does not cause rotation of the sleeve holder.

In Example 18, the valve hub assembly of Example 17, wherein the tightening port further includes a sleeve gripper capable of engagement with the sleeve holder.

In Example 19, the valve hub assembly of Example 16, further including a suture pad engaged with the distal end of the valve hub.

In Example 20, the valve hub assembly of Example 16, wherein the tightening port further includes a primary seal arranged within the hub cap and positioned adjacent the first lumen.

In Example 21, the valve hub assembly of Example 16, wherein the hub cap comprises a first portion, a second portion, and a transition wall defining a transition between the first portion and the second portion, and wherein the radially expandable seal is arranged within the first portion and adjacent the transition wall.

In Example 22, the valve hub assembly of Example 21, wherein the lock nut is configured to axially move the pusher against the radially expandable seal such that the radially expandable seal expands radially to seal against an inner surface of the hub cap.

In Example 23, the valve hub assembly of Example 16, wherein the radially expandable seal is a Tuohy seal.

In Example 24, the valve hub assembly of Example 16, wherein the hub cap has a plurality of protrusions extending from a proximal end of the hub cap, the valve hub has a collar with a plurality of openings, and the plurality of protrusions of the hub cap are engage with the plurality of openings such that rotation of the hub cap relative to the valve hub is inhibited.

In Example 25, a delivery system includes: a sheath and a hemostasis valve assembly. The sheath has a proximal end and a distal end and is configured for insertion through a blood vessel. The hemostasis valve hub assembly is engaged with the proximal end of the sheath and includes a tightening port arranged at the proximal end of a hemostasis valve hub. The tightening port includes: a hub cap engaged with a proximal end of the hemostasis valve hub, the hub cap having a first portion and a second portion and defining a lumen; a radially expandable seal positioned within the first portion; a pusher engaged with the radially expandable seal and at least partially disposed within the hub cap; and a lock nut arranged around the pusher and the hub cap and configured for pressing the pusher against the radially expandable seal.

In Example 26, the delivery system of Example 25, wherein the hub cap further includes a transition wall defining a transition between the first portion and the second portion, wherein the radially expandable seal is positioned adjacent the transition wall.

In Example 27, the delivery system of Example 25, wherein the lock nut is configured for axially pressing the pusher against the radially expandable seal.

In Example 28, the delivery system of Example 25, wherein the hemostasis valve hub includes a first arm and a second arm, and wherein a safety cap is engaged with the second arm and the tightening port is engaged with the first arm.

In Example 29, the delivery system of Example 25, wherein the hub cap has a plurality of protrusions extending from a proximal end of the hub cap, and the hemostasis valve hub has a collar with a plurality of openings, and the plurality of protrusions of the hub cap are engaged with the plurality of openings such that rotation of the hub cap relative to the hemostasis valve hub is inhibited.

In Example 30, a method for positioning and securing the positioning of a medical device includes assembling a hemostasis valve hub onto a sheath. The method further includes arranging a tightening port at a proximal end of the hemostasis valve hub, the tightening port including: a hub cap engaged with the proximal end of the homeostasis valve hub and the hub cap defining a lumen for receiving a radially expandable seal, a pusher engaged with the radially expandable seal and at least partially disposed within the hub cap, and a lock nut arranged around the pusher and hub cap. The method further includes delivering the medical device through the hemostasis valve hub and the sheath, verifying the position of the medical device, tightening the tightening port of the hemostasis valve hub to press the pusher against the radially expandable seal, and securing a sterile sleeve with the tightening port.

In Example 31, the method of Example 30, wherein the tightening the tightening port of the hemostasis valve hub includes actuating the lock nut to cause pressing of the pusher against the radially expandable seal.

In Example 32, the method of Example 31, wherein the pressing of the pusher against the radially expandable seal causes radial expansion of the radially expandable seal to cause sealed engagement between the radially expandable seal and the hub cap.

In Example 33, the method of Example 30, wherein the hub cap has a plurality of protrusions extending from a proximal end of the hub cap, the hemostasis valve hub has a collar with a plurality of openings, and the plurality of protrusions of the hub cap are engaged with the plurality of openings such that rotation of the hub cap relative to the hemostasis valve hub is inhibited.

In Example 34, the method of Example 30, wherein the verification of the position of the medical device is conducted via fluoroscopy.

In Example 35, the method of Example 30, wherein the securing of the sterile sleeve with the tightening port further includes placing the sterile sleeve over a sleeve holder of the tightening port and arranging a sleeve gripper over the sterile sleeve and the sleeve holder.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a side view of an introducer sheath extending into a blood vessel, in accordance with embodiments of the present disclosure.

FIG. 2 illustrates a cross sectional view of a medical device positioned within a blood vessel, in accordance with embodiments of the present disclosure.

FIG. 3 illustrates a side view of a valve hub assembly coupled to a tightening port, in accordance with embodiments of the present disclosure.

FIG. 4 illustrates a cross sectional view of the valve hub assembly coupled to the tightening port of FIG. 3, in accordance with embodiments of the present disclosure.

FIG. 5 illustrates an enlarged view of the valve hub assembly of FIG. 3, in accordance with embodiments of the present disclosure.

FIG. 6A illustrates an enlarged view of a seal for use with the valve hub assembly of FIG. 3, in accordance with embodiments of the present disclosure.

FIG. 6B illustrates a side view of the seal of FIG. 6A, in accordance with embodiments of the present disclosure.

FIG. 7 illustrates an enlarged perspective view of a hub cap for use with the valve hub assembly of FIG. 3, in accordance with embodiments of the present disclosure.

FIG. 8 illustrates an enlarged perspective view of a seal for use with the valve hub assembly of FIG. 3, in accordance with embodiments of the present disclosure.

FIG. 9 illustrates an enlarged perspective view of a pusher for use with the valve hub assembly of FIG. 3, in accordance with embodiments of the present disclosure.

FIG. 10A illustrates an enlarged front perspective view of a lock nut for use with the valve hub assembly of FIG. 3, in accordance with embodiments of the present disclosure.

FIG. 10B illustrates an enlarged rear perspective view of the lock nut of FIG. 10A, in accordance with embodiments of the present disclosure.

FIG. 11 illustrates an enlarged side perspective view of a sleeve holder for use with the valve hub assembly of FIG. 3, in accordance with embodiments of the present disclosure.

FIG. 12 illustrates an enlarged side view of a suture pad for use with the valve hub assembly of FIG. 3, in accordance with embodiments of the present disclosure.

FIG. 13 is a flow chart for a method of securing a medical device positioning through the use of the valve hub assembly of FIG. 3, in accordance with embodiments of the present disclosure.

### DETAILED DESCRIPTION

Hemostasis valve hub assemblies facilitate insertion and positioning of one or more medical devices (e.g., catheters, guidewires) while also helping to prevent blood from leaking during a procedure. Certain embodiments of the present disclosure feature assemblies with components that assist with fixing the position of one or medical devices while maintaining a seal. Further, certain embodiments also provide devices that facilitate convenient repositioning of the one or more medical devices.

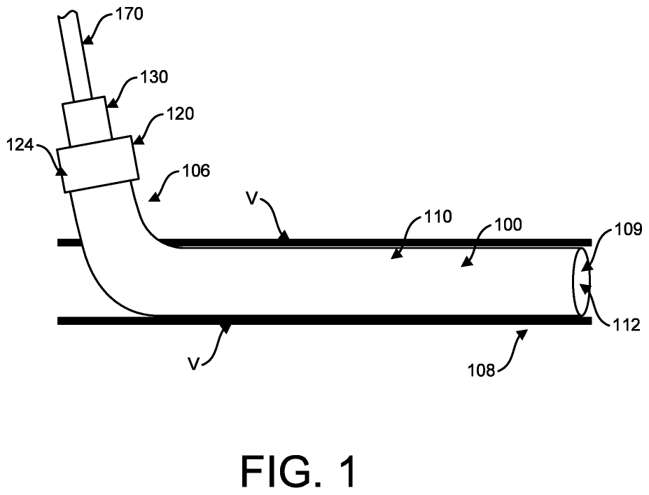

FIG. 1 illustrates a side cross sectional view of a blood vessel V with an introducer sheath 100, inserted at least partially into the blood vessel V. While the disclosure herein is made with reference largely to the introducer sheath 100, the disclosure may also apply to a repositioning sheath, as will be discussed further herein. In some embodiments, the introducer sheath 100 is used for facilitating the passage of various relatively large medical devices, such as a blood pump as will be described further herein, through the introducer sheath 100 and into the blood vessel V. Hence, the introducer sheath 100 may be referred to as a large bore introducer sheath. The introducer sheath 100 comprises a proximal end 106 and a distal end 108 that is opposite the proximal end 106. The introducer sheath 100 includes a proximal opening (not shown) adjacent the proximal end 106 and a distal opening 109 adjacent the distal end 108. A body portion 110 of the introducer sheath 100 extends between the proximal end 106 and the distal end 108, and the body portion 110 defines a lumen 112 of the introducer sheath 100. The introducer sheath 100 may be formed by various polymeric or metallic materials. In further embodiments, the introducer sheath 100 may comprise an additional surface coating. The surface coating may include, but is not limited to, silicone, PET, or an applicable polymer.

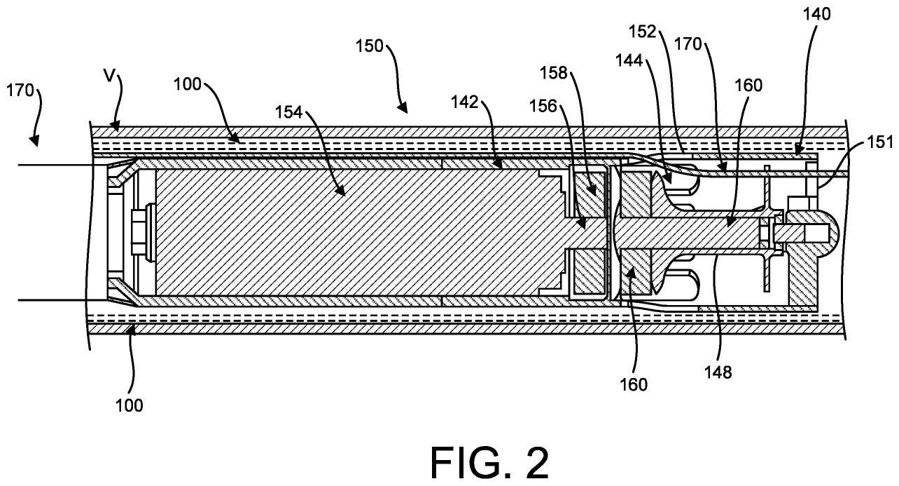

A hemostasis valve hub 120 (hereinafter “hub 120” for brevity) is commonly included at the proximal end 106 and over the proximal opening of the introducer sheath 100. The hub 120 is configured for hemostasis by, for example, helping to prevent blood from leaking out of the introducer sheath 100 during use. For example, a medical device such as a catheter 170 may be inserted through the hub 120 and the introducer sheath 100 and into the blood vessel V, and the hub 120 may maintain hemostasis between the catheter 170, the introducer sheath 100, and the external surroundings. In some embodiments, the catheter 170 may couple to a medical device such as the blood pump 150 shown in FIG. 2. After insertion of the catheter 170, fixation of the axial and radial position of the catheter 170 may be desired to ensure that the catheter 170 (and any coupled medical device) remains in the proper position during use. It may also be desired for the operator to reposition the catheter 170 (and any coupled medical device) after insertion. As such, the hub 120 may comprises a tightening port 130 composed of several components within the hub 120 that provides for the fixation of the catheter 170 with respect to the hub 120 and blood vessel V, as will be described further herein. The hub 120 and tightening port 130 may also allow for the repositioning of the catheter 170 with respect to the hub 120 and blood vessel V.

FIG. 2 illustrates a cross-sectional view of the introducer sheath 100 of FIG. 1 after insertion of a medical device, such as the blood pump 150, into the introducer sheath 100. As noted above, a catheter such as the catheter 170 may be coupled to the proximal end of the blood pump 150 and extend outside the blood vessel V and the introducer sheath 100. The blood pump 150 generally includes an impeller assembly housing 140 and a motor housing 142. In some embodiments, the impeller assembly housing 140 and the motor housing 142 may be integrally or monolithically constructed. The impeller assembly housing 140 carries an impeller assembly 144 therein. The impeller assembly 144 includes an impeller shaft 146 and an impeller 148 that rotates relative to the impeller assembly housing 140 to drive blood through the blood pump 150. More specifically, the impeller 148 causes blood to flow from a blood inlet 151 formed on the impeller assembly housing 140, through the impeller assembly housing 140, and out of a blood outlet 152 formed on the impeller assembly housing 140. In some embodiments the impeller shaft 146 and the impeller 148 may be integrally formed, and in other embodiments the impeller shaft 146 and the impeller 148 may be separate components. As shown in FIG. 2, the inlet 151 may be formed on an end portion of the impeller assembly housing 140 and the outlet 152 may be formed on a side portion of the impeller assembly housing 140. In other embodiments, the inlet 151 and/or the outlet 152 may be formed on other portions of the impeller assembly housing 140. In some embodiments, the impeller assembly housing 140 may couple to a distally extending cannula, and the cannula may receive and deliver blood to the inlet 151.

With continued reference to FIG. 2, the motor housing 142 carries a motor 154, and the motor 154 is configured to rotatably drive the impeller 148 relative to the impeller assembly housing 140. In the illustrated embodiment, the motor 154 rotates a drive shaft 156, which is coupled to a driving magnet 158. Rotation of the driving magnet 158 causes rotation of a driven magnet 160, which is connected to the impeller assembly housing 140. More specifically, in embodiments incorporating the impeller shaft 146, the impeller shaft 146 and the impeller 148 are configured to rotate with the driven magnet 160. In other embodiments, the motor 154 may couple to the impeller assembly housing 140 via other components. While the introducer sheath 100 is illustrated above with the use of the blood pump 150, various other medical devices may be used in conjunction with the introducer sheath 100 and the hemostasis valve hub 120.

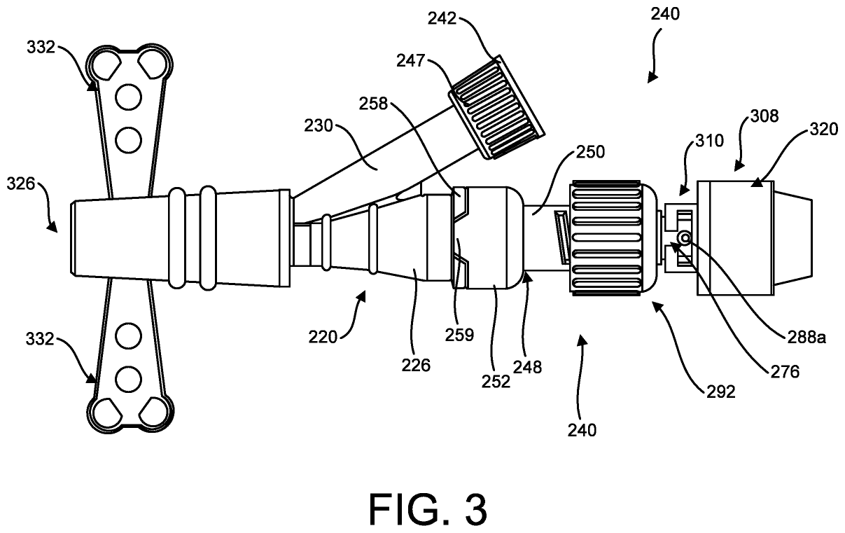

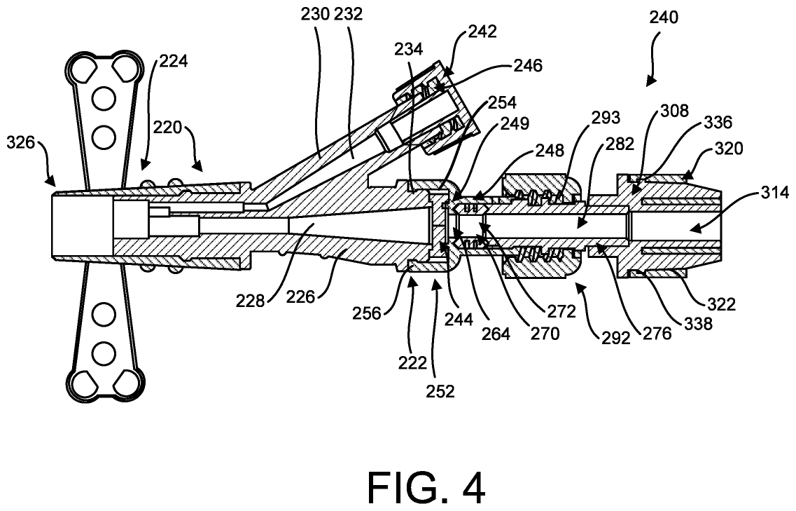

FIGS. 3 and 4 illustrate an embodiment of a hemostasis valve hub assembly having a hemostasis valve hub 220 (hereinafter “the hub 220” for brevity), a port 240 (e.g., a tightening port 240), and a suture pad 326 coupled together. Illustratively, the hub 220 is composed of a plurality of components including a safety cap 242 and a primary seal 244 (shown in FIG. 4). The tightening port 240 is composed of a plurality of components including a radially expandable seal or Tuohy seal 270 (FIG. 4), a hub cap 248, a pusher 276 engaged with the hub cap 248, a lock nut 292 engaged with the pusher 276, a sleeve holder 308 arranged at least partially over the pusher 276, and a sleeve gripper 320 disposed around the sleeve holder 308.

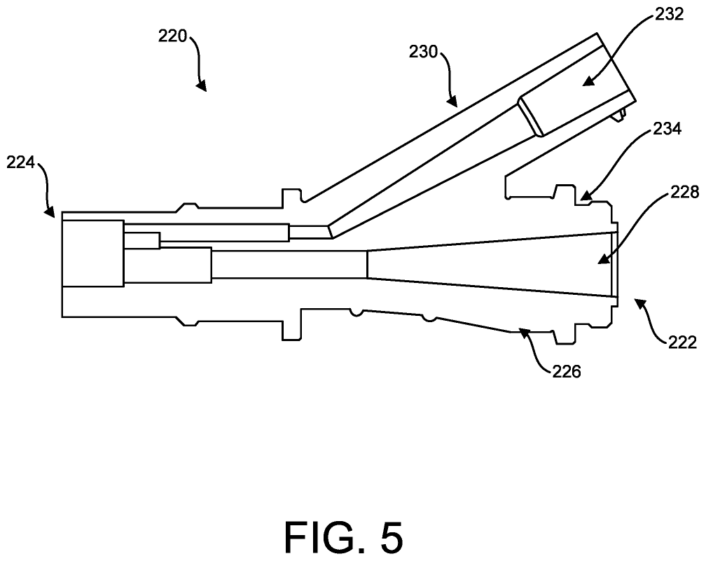

With reference to FIGS. 3 and 4 and the enlarged view of FIG. 5, the hub 220 will be described further herein. As illustrated, the hub 220 includes a proximal end 222 and a distal end 224. The hub 220 further includes a first arm 226 having a first lumen 228 extending between the proximal end 222 and the distal end 224, and a second arm 230 having a second lumen 232 extending between the proximal end 222 and the distal end 224. As illustrated, the first lumen 228 and the second lumen 232 are radially spaced from one another for a majority of a length of the hub 220, however, the first and second lumens 228, 232 may join together at the distal end 224 of the hub 220. In other embodiments, the first and second lumens 228, 232 may remain separate and connect with separate lumens in a sheath, for example, a dual lumen sheath. Hub 220 thus allows for separate tools to be simultaneously inserted through the first lumen 228 and the second lumen 232 of the hub 220 and then simultaneously inserted through the hub 220 and into the blood vessel V. For example, the first lumen 228 may be used for the reception of larger medical devices (e.g., a sheath, a catheter, a blood pump) while the second lumen 232 may be used for receiving smaller tools such as a guidewire.

Further, as illustrated in FIG. 3, the hub 220 includes a collar 258 extending from the proximal end 222 and having at least one cut out 259 extending within the collar 258. The collar 258 and at least one cut out 259 may be configured for engagement with the hub cap 248 as will be described further herein.

With reference to FIGS. 3, 4, and 6-12, the components of the hub 220 and tightening port 240 will be described further. For example, as illustrated in FIGS. 3 and 4, the safety cap 242 is engaged with a proximal end of the second arm 230 of the hub 220. In some embodiments, the safety cap 242 is threadedly engaged with the second arm 230, however, various other securing mechanisms may be incorporated. For example, as illustrated in FIG. 4, the safety cap 242 may have a plurality of threads 246 on an inner surface of the safety cap 242 for engaging with the second arm 230 and helping to create a fluid tight seal between the second lumen 232 and the safety cap 242. Additionally, as illustrated in FIG. 3, the safety cap 242 may have a plurality of ribs 247 on an outer surface of the safety cap 242 for aiding the operator in gripping the safety cap 242 during removal or insertion of the safety cap 242 onto the second arm 230.

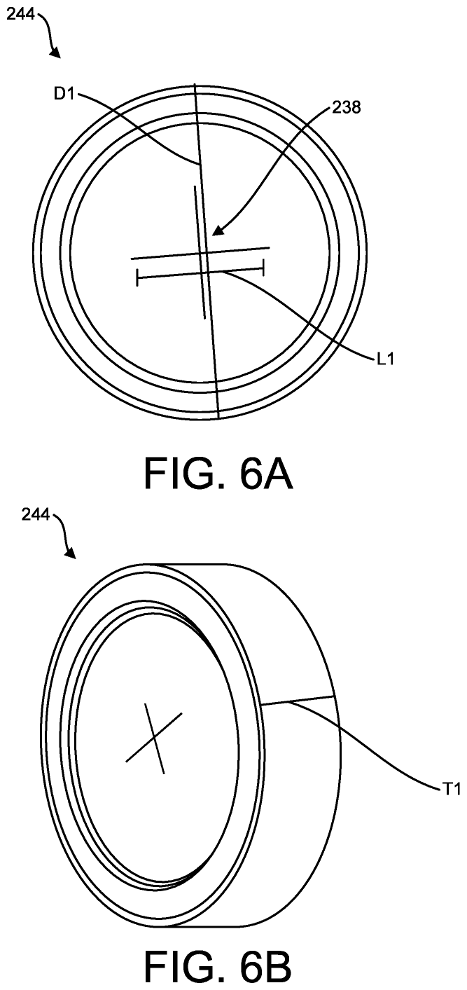

The primary seal 244 is illustrated as positioned against the first lumen 228 of the first arm 226 at the proximal end 222 of the hub 220. The primary seal 244 is configured for helping provide fluid tight seal around the medical device passing through the tightening port 240 and the hub 220, as will be described further herein. With reference to the enlarged view of FIG. 6A, the primary seal 244 is defined by a generally circular shape with a diameter D1. The diameter D1 may have a value of between approximately 8 mm and approximately 11 mm. For example, in some embodiments, the value of the diameter D1 may be approximately 9 mm. Additionally, as illustrated best in FIG. 6A, the primary seal 244 may have a thickness T1. The thickness T1 may have a value of between approximately 1.5 mm and 2.5 mm. For example, in some embodiments the thickness T1 is approximately 2 mm. With reference specifically to FIG. 6A, the primary seal 244 may have a partial cross slit 238 having a length L1 of approximately 4.5 mm. The primary seal 244 may be composed of silicone, or various other suitable materials like polymers (e.g., thermoset polymers such as thermoset elastomer (TSE) polymers and rubber such as silicone rubber).

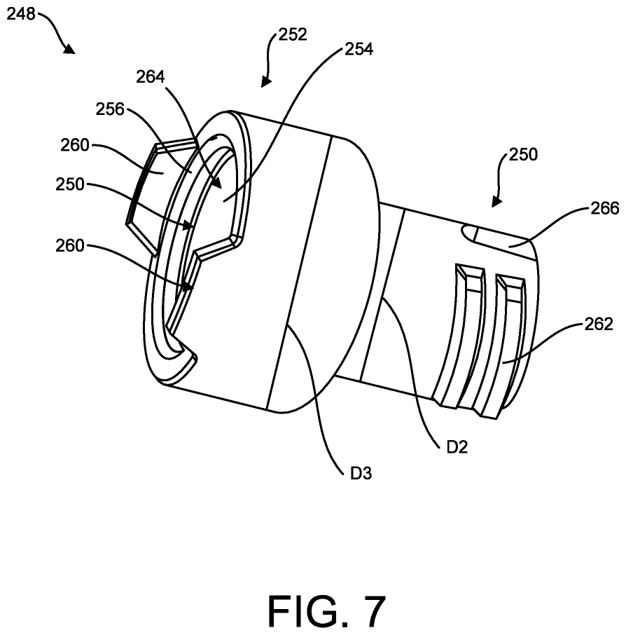

As illustrated in FIGS. 3 and 4, the hub cap 248 may be engaged with the proximal end 222 of the hub 220. With reference specifically to the enlarged view of FIG. 7, the hub cap 248 may include a first portion 250 having a diameter D2 and a second portion 252 having a diameter D3 that is larger than the diameter D2. For example, the value of diameter D2 may be between approximately 9 mm and approximately 11 mm and the value of diameter D3 may be between approximately 12.5 mm and approximately 15 mm. The hub cap 248 may also include a transition wall 249 defining a transition between the first portion 250 and the second portion 252. The second portion 252 of the hub cap 248 is configured for arrangement over the proximal end 222 of the hub 220, radial engagement with the hub 220, and for receiving the primary seal 244.

More specifically and as illustrated in FIG. 4, the second portion 252 has a rib 256 extending from an inner surface 254 of the second portion 252 which may engage with the groove 234 of the hub 220. The engagement between the rib 256 and the groove 234 may be facilitated by a press fit between the hub 220 and the hub cap 248. Additionally, as illustrated in the enlarged view of FIG. 7, the hub cap 248 may include at least one protrusion 260 extending from an end surface of the second portion 252 which may be received within the cut outs 259 of the collar 258 of the hub 220. For example, this is illustrated in FIG. 3, where the protrusions 260 are illustrated engaged with the cut outs 259. In this way, after engagement of the second portion 252 and the hub 220, the hub cap 248 is inhibited from rotation relative to the hub 220. As will be described below, inhibiting the hub cap 248 from rotation aids in inhibiting rotation of the medical device from causing the tightening port 240 to rotate during assembly.

With continued reference to FIG. 7, the first portion 250 comprises a plurality of threaded features 262 which may be configured for engagement with the lock nut 292 (FIG. 4), as will be described further herein. Additionally, the hub cap 248 comprises a lumen 264 extending through both the first portion 250 and the second portion 252 which allows for the reception of the primary seal 244 within the second portion 252 and the radially expandable seal 270 (FIG. 4) within the first portion 250, as will be described further herein. Additionally, the first portion 250 of the hub cap 248 may include an opening 266 which may be configured for receiving a protrusion of the pusher 276, as will be described further herein.

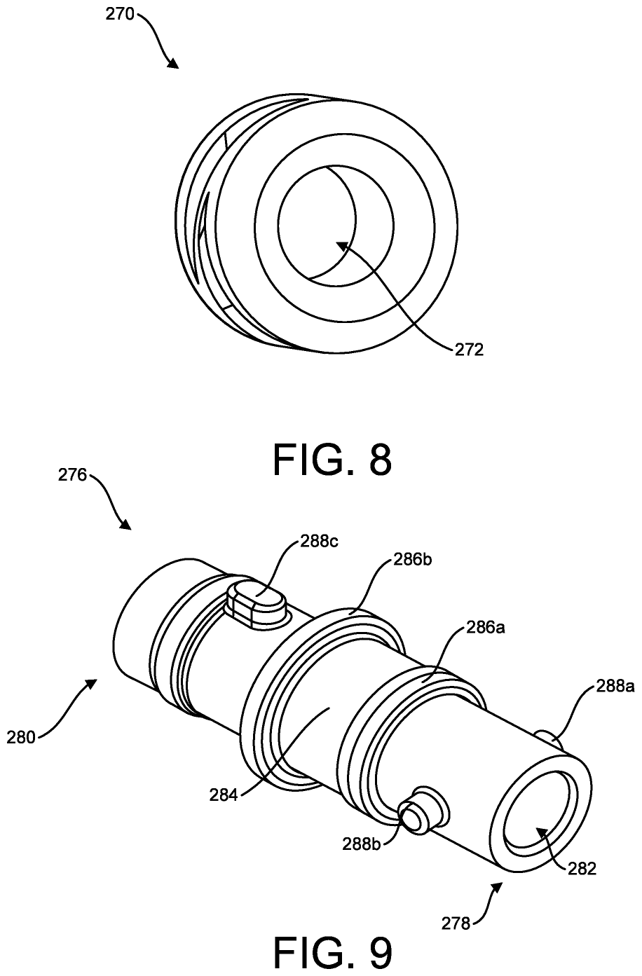

With reference again to FIGS. 3 and 4, the tightening port 240 includes the radially expandable seal 270 arranged within the first portion 250 (FIG. 3) of the hub cap 248. FIG. 8 illustrates a perspective enlarged view of the radially expandable seal 270. The radially expandable seal 270 is defined by a generally cylindrical shape and has a lumen 272 extending therethrough. The radially expandable seal 270 may be a Tuohy seal, such that axial compression of the seal 270 causes radial expansion of the seal 270 to engage with surfaces of which the seal 270 is positioned against. In these embodiments, this creates a fluid tight seal around the medical device passing through the tightening port 240 within the hub cap 248. The seal 270 thus also fixes the position of the medical device with respect to the hub 220 and the tightening port 240.

As previously referenced, the first portion 250 of the hub cap 248 may be engaged with the pusher 276. With reference to FIGS. 3, 4, and 9, the pusher 276 will be described further herein. The pusher 276 may be received at least partially within the hub cap 248 and engaged with the radially expandable seal 270. FIG. 9 illustrates a perspective view of the pusher 276. As illustrated, the pusher 276 has a cylindrical configuration with a proximal end 278 and a distal end 280 and a lumen 282 extending therethrough. The pusher 276 has a body that is shaped—or coupled to various components—to provide the various functions described herein, including engaging with other components of the valve hub assembly to help provide a seal. The body of the pusher 276 is defined by an outer surface 284 having a plurality of ribs 286 and a plurality of protrusions 288 extending from the outer surface 284. More specifically, the plurality of ribs 286 includes a first rib 286a arranged toward the proximal end 278 of the pusher 276 and a second rib 286b arranged toward the distal end 280 of the pusher 276. The plurality of protrusions 288 includes a first protrusion 288a and a second protrusion 288b extending radially outward from the outer surface 284 and positioned opposite one another along a circumference of the pusher 276. In other words, the first and second protrusions 288a, 288b may be spaced circumferentially by approximately 180 degrees. First and second protrusions 288a, 288b are arranged proximally relative to the first rib 286a.

The plurality of protrusions 288 further includes a third protrusion 288c that is arranged distally of the second rib 286b and positioned circumferentially offset relative to the first and second protrusions 288. As will be described further herein, the plurality of ribs 286 and the plurality of protrusions 288 may be configured for engaging and securing the pusher 276 with the hub cap 248, the lock nut 292, and the sleeve holder 308.

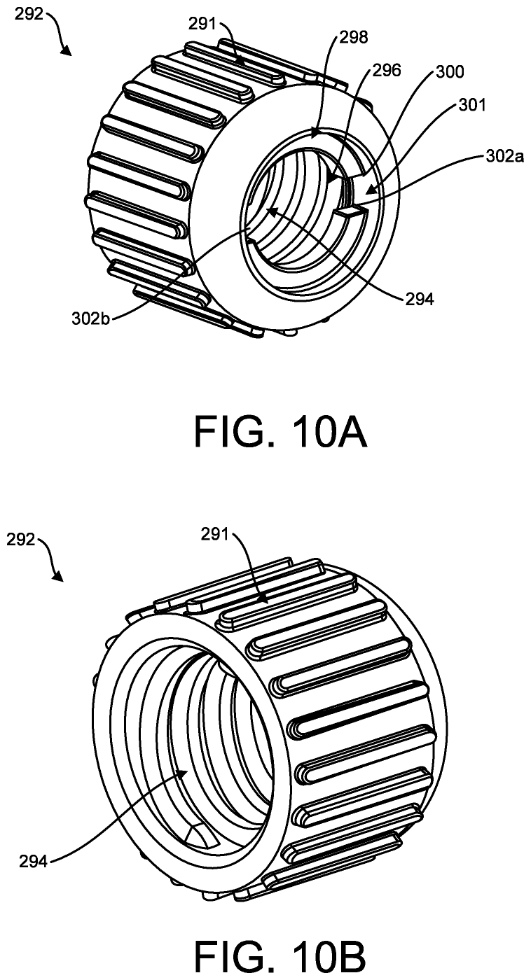

With reference to FIG. 4 and the enlarged view of the FIGS. 10A and 10B, the lock nut 292 will be described further herein. As illustrated, the lock nut 292 has a generally cylindrical profile with a lumen 294 disposed therein. Additionally, the lock nut 292 includes a plurality of ribs 291 arranged around an outer surface of the lock nut 292 which increase the case with which the operator may grasp or grip the lock nut 292 and rotate the lock nut 292 during use. Further, the lock nut 292 includes a plurality of threaded features 296, or ribs, extending from an inner surface 301 of the lock nut 292, which may be used for engagement with the pusher 276 and/or the hub cap 248, as will be described further herein. Additionally, with reference specifically to FIG. 10A, the lock nut 292 includes a collar 298 extending radially inward from the inner surface 301 of the lock nut 292. The collar 298 may have at least one cut out 302, or illustratively two cut outs 302a, 302b, which may be used for the engagement between the lock nut 292 and the pusher 276.

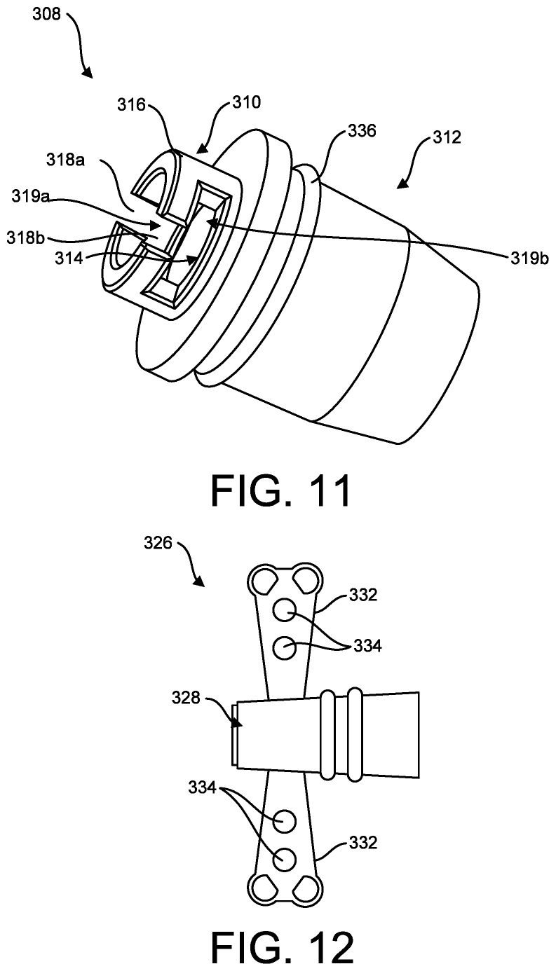

Further, with reference to FIGS. 3 and 4, the sleeve holder 308 may also be received over a portion of the pusher 276, illustratively over the proximal end 278 of the pusher 276. Notably, as shown, sleeve holder 308 does not engage lock nut 292, thus allowing sleeve holder 308 and lock nut 292 to move independently of each other. With reference to the enlarged view of FIG. 11, the sleeve holder 308 will be described further herein. The sleeve holder 308 is defined by an engagement portion 310 configured for engagement with the pusher 276 and a second portion 312 extending proximally from the engagement portion 310 and having a lumen 314 extending therethrough. More particularly, the engagement portion 310 is defined by a collar 316 extending outwardly from the second portion 312. The collar 316 comprises two cut outs 318a and 318b which may each extend into openings 319a, 319b defined by walls of the collar 316. As illustrated, each of the openings 319a and 319b is generally rectangular in shape, however various other configurations may be incorporated. As will be described further herein, the openings 319a and 319b are configured for receiving protrusions of the pusher 276 to facilitate engagement of the sleeve holder 308 and the pusher 276. Additionally, the sleeve holder 308 comprises a rib 336 extending circumferentially around the second portion 312. The second portion 312 may be configured for receiving the sleeve gripper 320. The sleeve gripper 320 is cylindrical and defines a lumen 322 extending therethrough. The sleeve gripper 320 may additionally include a groove 338 (FIG. 4) which may engage with the sleeve holder 308 when the second portion 312 of the sleeve holder 308 is received within the lumen 322 of the sleeve gripper 320. In this way, second portion 312 is received within the lumen 322 of the sleeve gripper 320 when engaged.

The suture pad 326 is configured to be engaged with the distal end 224 of the hub 220 and comprises a main portion 328 having a cylindrical configuration and a lumen 330 extending therethrough and for receiving the proximal end 222 of the hub 220. Further, the suture pad 326 includes at least two extensions 332 extending radially outward from the main portion 328. The two extensions 332 define planar surfaces having a plurality of openings 334 extending therethrough. The two extensions 332 and the openings 334 extending therethrough allow for securing of the suture pad 326 onto a subject. For example, the openings 334 may be used for suturing of the suture pad 326 to the skin of the patient. However, various other mechanisms for securing the hub 220 to the patient may be incorporated.

With reference to FIGS. 3 and 4, assembly and engagement of the components of the suture pad 326 and the tightening port 240 onto the hub 220 will be described herein. As illustrated, the suture pad 326 is engaged with the distal end 224 of the hub 220. More particularly, the main portion 328 of the suture pad 326 is arranged over the distal end 224 of the hub 220 such that the distal end 224 of the hub 220 is received within the lumen 330 of the suture pad 326. Additionally, the safety cap 242 is illustrated as engaged with the second arm 230 of the hub 220. As previously described, the safety cap 242 includes the plurality of threads 246 for engagement with the second arm 230 of the hub 220, such that a portion of the safety cap 242 is arranged around the second arm 230 and a portion of the safety cap 242 is arranged within the lumen 232 of the second arm 230. This engagement of the safety cap 242 with the hub 220 allows for a hemostatic seal within the second arm 230.

With reference still to FIGS. 3 and 4, the primary seal 244 is positioned against the proximal end 222 of the hub 220, and more particularly against the first arm 226 of the hub 220. Further, the partial cross slits 238 are aligned with the first lumen 228 of the first arm 226 such that a continuous lumen is formed through the first arm 226 and the primary seal 244 so that a medical device may be inserted through the hub 220 and the partial cross slit 238 of the primary seal 244. Further, the hub cap 248 is arranged over the primary seal 244 and engaged with the proximal end 222 of the hub 220. As previously described, the groove 234 of the hub 220 engages with the rib 256 of the hub cap 248 to secure coupling between the hub 220 and the hub cap 248. Thus, a hemostatic seal may be formed between the catheter 170 and the primary seal 244, and as such, the hub 220. Even further, the protrusions 260 of the hub cap 248 may be arranged within the cut outs 259 formed of the collar 258 extending around the first arm 226 of the hub 220. In this way, once the hub cap 248 is engaged with the hub 220, any rotation of the hub cap 248 is inhibited as the protrusions 260 will be positioned against the edges of the cut outs 259 of the collar 258. In other words, the protrusions 260 engage with the cut outs 259 to ensure that during tightening or loosening of the lock nut 292 or repositioning of any medical device extending through the hub 220, the hub cap 248 is unable to rotate and/or be removed from the hub 220.

With reference still to FIGS. 3 and 4, the radially expandable seal 270 is arranged within the first portion 250 of the hub cap 248 and against the transition wall 249 of the hub cap 248. During assembly, the radially expandable seal 270 may be positioned within the first portion 250 of the hub cap 248 and the pusher 276 may be used to axially compress the radially expandable seal 270 against the transition wall 249 during engagement of the pusher 276 with the hub cap 248. More specifically, the distal end 224 of the pusher 276 may be placed into the first portion 250 of the hub cap 248 and the third protrusion 288c may engage with the opening 266 of the hub cap 248 to restrict rotation of the pusher 276 relative to the hub cap 248. The pusher 276 may be pushed into hub cap 248 until the second rib 286b is arranged adjacent to the proximal end 222 of the hub cap 248. Once the pusher 276 is engaged with the hub cap 248 in this manner, the engagement of the pusher 276, the radially expandable seal 270 and the hub cap 248 may be secured with the lock nut 292. The lock nut 292 may be arranged over the pusher 276 until the lock nut 292 is arranged distally relative to the rib 286a that is toward the proximal end 278 of the pusher 276. More particularly, when the lock nut 292 is arranged over the pusher 276, the first and second protrusions 288a, 288b of the pusher 276 may be inserted through the cut outs 302a, 302b of the collar 298 such that the first and second protrusions 288a, 288b may extend proximally relative to the cut outs 302a, 302b. As previously described, the lock nut 292 is arranged such that the rib 268a is also arranged proximally relative to the cut outs 302a, 302b and thus the collar 298. Once arranged over the pusher 276, the threads 294 of the hub cap 248 engage with the threaded features 262 of the hub cap 248. Thus, the lock nut 292 can be rotated to engage with the hub cap 248 such that the hub cap 248 moves distally and thus actuates the pusher 276 distally and causes the pusher 276 to push against the radially expandable seal 270. This can cause the radially expandable seal 270 to expand and seal against the inner surface of the hub cap 248 as well as against the medical device passing through the seal 170. However, the engagement of the third protrusion 288c within the opening 266 of the hub cap 248 helps ensures that the rotation of the lock nut 292 only pushes the pusher 276 distally to a predetermined extent. In other words, the distal movement of the pusher 276 against the expandable seal 270 can continue until the protrusions 288c against the surface of the opening 266.

Thus, a hemostatic seal may be created between the medical device extending within the radially expandable seal 270 and the hub cap 248. In addition, the expandable seal 270 may secure the position of the medical device with respect to the hub 220, for example helping to inhibit the medical device from moving axially or radially within the hub 220. In one example, the expansion of expandable seal 270 as described herein may secure catheter 170 within hub 220, helping to prevent movement of the catheter axially within the hub 220. In another example, the expansion of expandable seal 270 as described herein may secure catheter 170 within hub 220, helping to prevent movement of the catheter axially within the hub 220 and in turn securing the position of blood pump 150 coupled to catheter 170 within the patient's vasculature.

To reposition the medical device, the lock nut 292 is loosened by rotating the lock nut 292 such that the lock nut 292 moves proximally relative to the pusher 276 to release engagement between the pusher 276 and the lock nut 292. This releases at least some compression between the pusher 276 and the expandable seal 270 which causes the pusher 276 to move away from the expandable seal 270, releasing the expandable seal 270 against the inner surface of the hub cap 248 as well as against the medical device passing through the seal 170, and allowing the medical device to be repositioned. After repositioning is complete, the lock nut 292 may be retightened, securing the medical device in position once again.

Further, with reference still to FIGS. 3 and 4, the sleeve holder 308 is positioned over the proximal end 278 of the pusher such that the engagement portion 310 may be engaged with the first and second protrusions 288a, 288b of the pusher 276. After first and second protrusions 288a, 288b are inserted through the openings 319 of sleeve holder 308, the sleeve holder 308 may be rotated to cause engagement of the first and second protrusions 288a, 288b with walls of the collar 316 of the engagement portion 310.

Lastly, the sleeve gripper 320 may then be positioned over the second portion 312 of the sleeve holder 308. As illustrated, the rib 336 of the sleeve holder 308 may engage with the groove 338 (FIG. 4) of the sleeve gripper 320 to ensure secured coupling of the sleeve gripper 320 and the sleeve holder 308. This engagement may be done after the positioning of a sterile sleeve over the sleeve gripper 320 to fix the positioning of the sleeve holder 308.

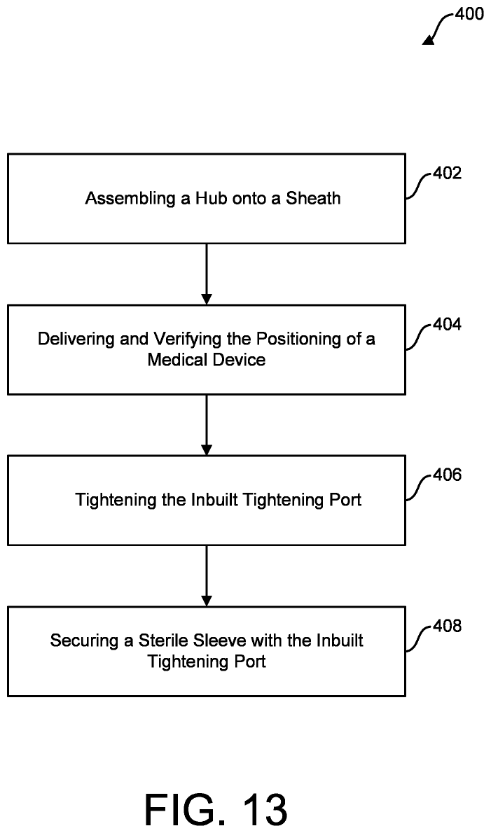

As described throughout, a variety of medical devices may be inserted through the hub 220 for insertion into the introducer sheath 100. For example, the catheter 170, blood pump 150, and/or various other medical devices may be inserted through the hub 220 for positioning within the blood vessel V. In some embodiments, it may be desired for the introducer sheath 100 to be removed and replaced with a repositioning sheath. In these embodiments, the previously described hub 220 and the tightening port 240 may be arranged on a proximal end of the repositioning sheath, although the dimensions of the hub 220 and tightening port 240 or their components may be different when used with a repositioning sheath as compared to an introducer sheath. The tightening port 240 in particular may provide the advantage of allowing for repositioning of the catheter 170, blood pump 150, and/or various other medical devices may be inserted through the hub 220 and associate sheath, by allowing for loosening and retightening of the tightening port 240 to occur after initial positioning of the medical devices. In particular, the tightening port 204 may allow for repositioning of a medical device while avoiding twisting of an associated sterile sleeve due to the sleeve holder 308 being independent of the lock nut 292. This allows the user to tighten and loosen the lock nut 292, allowing for repositioning of the medical device, and then retighten the lock nut 292. The overall process will be described further with reference to FIG. 13.

FIG. 13 is a flow chart illustrating a method 400 of locking the positioning of a medical device, for example the catheter 170, extending through the hub 220 and tightening port 240. While the description is described herein with reference to the catheter 170, various other medical devices may be used with the method described herein. For example, the method 400 may be applied for use with the catheter 170 coupled with the blood pump 150.

At block 402, the method 400 first includes assembling the hub 220 having the tightening port 240 onto a sheath. In some embodiments, the sheath may be the introducer sheath 100, however, in other embodiments the sheath may be a repositioning sheath. The method 400 will be described herein with reference to the introducer sheath 100. When the hub 220 is assembled onto the sheath 100, the tightening port 240 may be fully assembled as illustrated in FIG. 3, however the lock nut 292 may be in a loosened configuration.

At block 404, the method 400 further includes delivering a medical device, for example the catheter 170, through the hub 220 and thus the tightening port 240, and the introducer sheath 100. This step additionally includes verifying the positioning of the catheter 170 within the patient's body. This may be completed through the use of fluoroscopy or various other imaging mechanisms. While described herein as the catheter 170, in some embodiments, the blood pump 150 may be delivered through the hub 220 and the introducer sheath 100 and the positioning of the blood pump 150 within the patient's body is verified.

Further, at block 406, the method 400 includes tightening the tightening port 240. In embodiments, this step may further include actuating the lock nut 292 to engage with the pusher 276 to axially compress the pusher 276 against the radially expandable seal 270. The radially expandable seal 270 thus expands into sealed engagement with the inner surface of the hub cap 248. In this way, a fluid tight seal around the catheter 170 is created within the hub cap 284, in addition to the fluid tight seal provided by primary seal 244 at the proximal end 222 of the hub 220. As described above, the actuation of expandable seal 270 fixes the position of the medical device relative to the hub 220 and sheath 100. Additionally, the method 400 may also include repositioning the catheter 170, and/or the blood pump 150, until the catheter 170 and/or blood pump 150 is in the desired position. Once the blood pump 150 is in the proper position, the lock nut 292 may be rotated as described previously in order to push the pusher 276 distally and into engagement with the expandable seal 270 to secure the position of the catheter 170. Due to the independent arrangement between the sleeve holder 308 and the lock nut 292, rotation of the lock nut 292 does not cause rotation of the sleeve holder 308, and therefore the sleeve holder 308 and the sterile sleeve remain in place and need not be adjusted by the operator.

At block 408, the method 400 further includes securing a sterile sleeve with the tightening port 240. Prior to this step, the sterile sleeve has already been secured between the sleeve gripper 320 and the sleeve holder 308, such that the sleeve gripper 320, sleeve holder 308 and sterile sleeve are a pre-assembled assembly. Thus, the step at block 408 includes attaching the pre-assembled sleeve gripper 320, sleeve holder 308 and sterile sleeve onto the tightening port 240. As noted above, the sleeve holder 308 does not engage lock nut 292, meaning that the lock nut 292 can be manipulated without manipulating the sleeve holder 308. In other words, the lock nut 292 can be tightened, loosened, and the catheter 170 and/or blood pump 150 may be repositioned without affecting the sleeve holder 308 or the attached sterile sleeve. Likewise, the lock nut 292 may be retightened after repositioning of the catheter 170 and/or blood pump 150 without affecting the sleeve holder 308 or the attached sterile sleeve.

The ability to tighten the tightening port 240, and more specifically the radially expandable seal 270, and then subsequently secure the sleeve with tightening port, provides the advantage of accounting for possible repositioning of the catheter 170 and/or the introducer sheath 100 before securing the sleeve. Because the movement of the catheter 170 may cause shifting of the blood pump from its desired position, or because the physician may elect to change the position of the blood pump after initial placement, being able to the tighten, loosening, and retighten the tightening port 240 prior to securing the sterile sleeve to the tightening port 240 reduces the chances that the sterile sleeve will be twisted or otherwise disfigured, and thus may reduce need to remove or reposition the sterile sleeve after repositioning the catheter 170 and/or blood pump.

While the embodiments and method are described with reference to the catheter 170 and the blood pump 150 for reception into the catheter 170, various other medical devices may be incorporated. Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

What is claimed is:

1. A valve hub assembly for use with a sheath, the valve hub assembly comprising:
a valve hub having a proximal end opposite a distal end, a first arm defining a first lumen, and a second arm defining a second lumen; and
a tightening port arranged at the proximal end of the valve hub, the tightening port including:
a hub cap engaged with the proximal end of the valve hub, the hub cap defining a third lumen for receiving a radially expandable seal,
a pusher engaged with the radially expandable seal and at least partially disposed within the hub cap, and
a lock nut arranged around the pusher and configured for tightening of the pusher against the radially expandable seal.

2. The valve hub assembly of claim 1, further comprising a sleeve holder for engaging the pusher without engaging the lock nut, wherein engagement of the sleeve holder and the pusher is configured such that rotation of the lock nut does not cause rotation of the sleeve holder.

3. The valve hub assembly of claim 2, wherein the tightening port further includes a sleeve gripper capable of engagement with the sleeve holder.

4. The valve hub assembly of claim 1, further comprising a suture pad engaged with the distal end of the valve hub.

5. The valve hub assembly of claim 1, wherein the tightening port further includes a primary seal arranged within the hub cap and positioned adjacent the first lumen.

6. The valve hub assembly of claim 1, wherein the hub cap comprises a first portion, a second portion, and a transition wall defining a transition between the first portion and the second portion, and wherein the radially expandable seal is arranged within the first portion and adjacent the transition wall.

7. The valve hub assembly of claim 6, wherein the lock nut is configured to axially move the pusher against the radially expandable seal such that the radially expandable seal expands radially to seal against an inner surface of the hub cap.

8. The valve hub assembly of claim 1, wherein the radially expandable seal is a Tuohy seal.

9. The valve hub assembly of claim 1, wherein the hub cap has a plurality of protrusions extending from a proximal end of the hub cap, the valve hub has a collar with a plurality of openings, and the plurality of protrusions of the hub cap are engage with the plurality of openings such that rotation of the hub cap relative to the valve hub is inhibited.

10. A delivery system for at least one medical device into a blood vessel, the delivery system comprising:
a sheath having a proximal end and a distal end and configured for insertion through the blood vessel; and
a hemostasis valve hub assembly engaged with the proximal end of the sheath, the hemostasis valve hub assembly includes a tightening port arranged at the proximal end of a hemostasis valve hub, the tightening port including:
a hub cap engaged with a proximal end of the hemostasis valve hub, the hub cap having a first portion and a second portion and defining a lumen,
a radially expandable seal positioned within the first portion,
a pusher engaged with the radially expandable seal and at least partially disposed within the hub cap, and
a lock nut arranged around the pusher and the hub cap and configured for pressing the pusher against the radially expandable seal.

11. The delivery system of claim 10, wherein the hub cap further includes a transition wall defining a transition between the first portion and the second portion, and wherein the radially expandable seal is positioned adjacent the transition wall.

12. The delivery system of claim 10, wherein the lock nut is configured for axially pressing the pusher against the radially expandable seal.

13. The delivery system of claim 10, wherein the hemostasis valve hub includes a first arm and a second arm, and wherein a safety cap is engaged with the second arm and the tightening port is engaged with the first arm.

14. The delivery system of claim 10, wherein the hub cap has a plurality of protrusions extending from a proximal end of the hub cap, and the hemostasis valve hub has a collar with a plurality of openings, and the plurality of protrusions of the hub cap are engaged with the plurality of openings such that rotation of the hub cap relative to the hemostasis valve hub is inhibited.

15. A method for positioning and securing the positioning of a medical device, the method comprising:
assembling a hemostasis valve hub onto a sheath;
arranging a tightening port at a proximal end of the hemostasis valve hub, the tightening port including: a hub cap engaged with the proximal end of the homeostasis valve hub and the hub cap defining a lumen for receiving a radially expandable seal, a pusher engaged with the radially expandable seal and at least partially disposed within the hub cap, and a lock nut arranged around the pusher and hub cap;
delivering the medical device through the hemostasis valve hub and the sheath;
verifying the position of the medical device;
tightening the tightening port of the hemostasis valve hub to press the pusher against the radially expandable seal; and
securing a sterile sleeve with the tightening port.

16. The method of claim 15, wherein the tightening the tightening port of the hemostasis valve hub includes actuating the lock nut to cause pressing of the pusher against the radially expandable seal.

17. The method of claim 16, wherein the pressing of the pusher against the radially expandable seal causes radial expansion of the radially expandable seal to cause sealed engagement between the radially expandable seal and the hub cap.

18. The method of claim 15, wherein the hub cap has a plurality of protrusions extending from a proximal end of the hub cap, the hemostasis valve hub has a collar with a plurality of openings, and the plurality of protrusions of the hub cap are engaged with the plurality of openings such that rotation of the hub cap relative to the hemostasis valve hub is inhibited.

19. The method of claim 15, wherein the verification of the position of the medical device is conducted via fluoroscopy.

20. The method of claim 15, wherein the securing of the sterile sleeve with the tightening port further includes placing the sterile sleeve over a sleeve holder of the tightening port and arranging a sleeve gripper over the sterile sleeve and the sleeve holder.
